(19) United States
(12) Patent Application Publication
(10) Pub. No.: US 2015/0359488 A1
(43) Pub. Date: Dec. 17, 2015

(54) ACTIVITY-SENSING GROUND PAD AND PAD ASSEMBLY HAVING THE SAME

(71) Applicants: Yeh-Liang Hsu, Taoyuan County (TW); SEDA CHEMICAL PRODUCTS CO., LTD., New Taipei City (TW)

(72) Inventors: YEH-LIANG HSU, Taoyuan County (TW); WEI-KUAN WANG, New Taipei City (TW); KAI-WEI CHANG, Miaoli County (TW); YU-WEI LIU, New Taipei City (TW); WEI-YI CHANG, Taipei City (TW)

(21) Appl. No.: 14/714,260

(22) Filed: May 16, 2015

(30) Foreign Application Priority Data
Jun. 16, 2014 (TW) 103120738

(51) Int. Cl. A61B 5/00; A61B 5/11

(52) U.S. Cl. CPC A61B 5/6892; A61B 5/1117; A61B 5/1113

(57) ABSTRACT

The present invention provides an activity-sensing ground pad for integrating with a monitoring apparatus to continuously monitor human activity in the home environment. The activity-sensing ground pad includes a modular base, a sensing layer arranged on the modular base, an insulating layer arranged on the sensing layer, and a conductive elastic layer formed to cover the insulating layer. Specifically, the modular base has a first assembling side with a plurality of first engagement portions thereon and a second assembling side opposite to the first assembling side with a plurality of second engagement portions thereon, wherein a first electrical connector is arranged on one of the engaging portions, and a second electrical connector is arranged on one of the engaging portions in correspondence with the first electrical connector.

### BACKGROUND OF THE INVENTION

1. Field of the Invention

The instant disclosure relates to a device for monitoring human activity; in particular, to an activity-sensing ground pad and a pad assembly including a plurality of activity-sensing ground pads which is used for indoor positioning, human activity monitoring, fall detecting, etc.

2. Description of Related Art

Falls in elderly people have been recognized as a major health problem in an aging population. Physical activity patterns, detecting the occurrence of falls, and recognizing body motion patterns inevitably leading to falls are not well understood due to the lack of systems which allow continuous monitoring of patients in an accurate, convenient, unobtrusive and socially acceptable manner. Hence touch sensor pads with integrated button functionality have been developed to create fall prevention or detection systems that address this concern.

In general, inside the touch sensor pad there is arranged a sensor module. The sensor module is configured to transmit a detection signal to a monitoring device (i.e. computer) when receiving an external force. Thereby, abnormal events can be quickly detected, and a nurse or a personal care attendant can track down elderly people and then aid them. If the touch sensor pads are widely used everywhere in people's homes and even big caring centers (pads are used at plenty of floors and there are some big or small chambers at each floor), even though the nurse can be noticed of a certain emergency via the monitoring device, it is still hard to immediately know the place where the emergency has happened and thus hard to deal with the emergency in good time.

A better way to improve this situation is to cut the original pad-covered range, which is bigger, into many smaller areas, and then to monitor each smaller area via a monitoring device to know whether there is an emergency happening After that, all information fed back from the monitoring device can be received by a central processing device. This might be a practicable solution. However, cutting the original pad-covered range into many smaller areas, though helping to precisely know where the emergency happened, also means there would be many more monitoring devices needed, which increases the cost.

The traditional touch sensor pads help the nurse to know an emergency has happened as soon as possible, but when used in a wide area, it is hard for the nurse to immediately know where the emergency happened and thus hard to deal with the emergency in good time.

### SUMMARY OF THE INVENTION

The object of the instant disclosure is to provide an activity-sensing ground pad and pad assembly including a plurality of activity-sensing ground pads to detect and track the human motion of the elderly, children, and persons with disabilities by an unconscious method.

In order to achieve the aforementioned objects, according to an embodiment of the instant disclosure, the activity-sensing ground pad includes a modular base, a sensing layer arranged on the modular base, an insulating layer arranged on the sensing layer, and a conductive elastic layer formed to cover the insulating layer. Specifically, the modular base has a first assembling side with a plurality of first engagement portions thereon and a second assembling side opposite to the first assembling side with a plurality of second engagement portions thereon, wherein a first electrical connector is arranged on one of the engaging portions, and a second electrical connector is arranged on one of the engaging portions in correspondence with the first electrical connector.

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 is an exploded view of an activity-sensing ground pad according to a preferred embodiment of the present invention;

FIG. 2 is an assembled view of the activity-sensing ground pad according to a preferred embodiment of the present invention;

FIG. 3 is a perspective view showing a modular base and a sensing layer;

FIG. 4 is a perspective view showing a use state of the activity-sensing ground pad;

FIG. 5 is a perspective view showing another use state of the activity-sensing ground pad;

FIG. 6 is an assembled view of an activity-sensing ground pad assembly according to a preferred embodiment of the present invention; and

FIG. 7 is a perspective view showing a use state of the activity-sensing ground pad assembly.

### DETAILED DESCRIPTION OF THE PREFERRED EMBODIMENTS

The aforementioned illustrations and following detailed descriptions are exemplary for the purpose of further explaining the scope of the instant disclosure. Other objectives and advantages related to the instant disclosure will be illustrated in the subsequent descriptions and appended drawings.

The present invention provides an activity-sensing ground pad which applies human motion sensing technologies to ground pad structure, and adapted to interlock with one another to meet the requirements of indoor spatial applications. Thereby, the human motion of the elderly, children, and persons with disabilities can be detected and tracked by an unconscious method.

Further, on the main body of the activity-sensing ground pad there are arranged a sensing layer and a conductive elastic layer on the sensing layer. The conductive elastic layer and the sensing layer can act together as a piezoresistive sensor to determine the user's activity state (i.e. walking state, standing state, sitting state, falling down state, etc.) according to the correlation of sensed-pressure-data and output resistance without affecting the user's daily life.

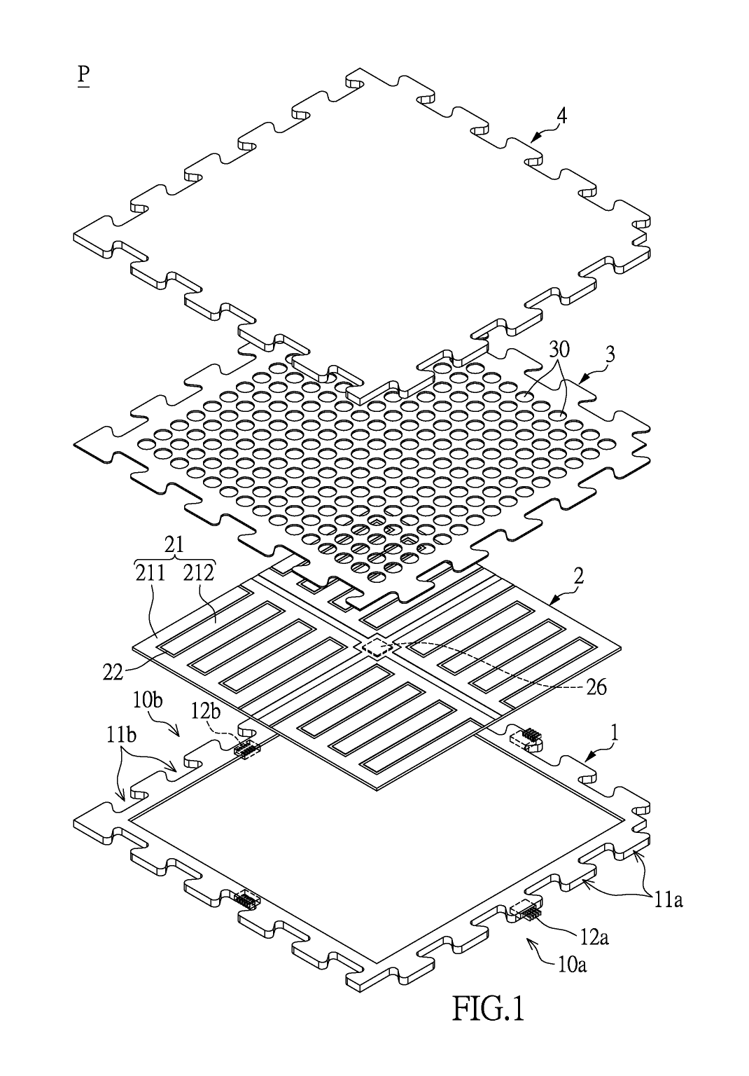

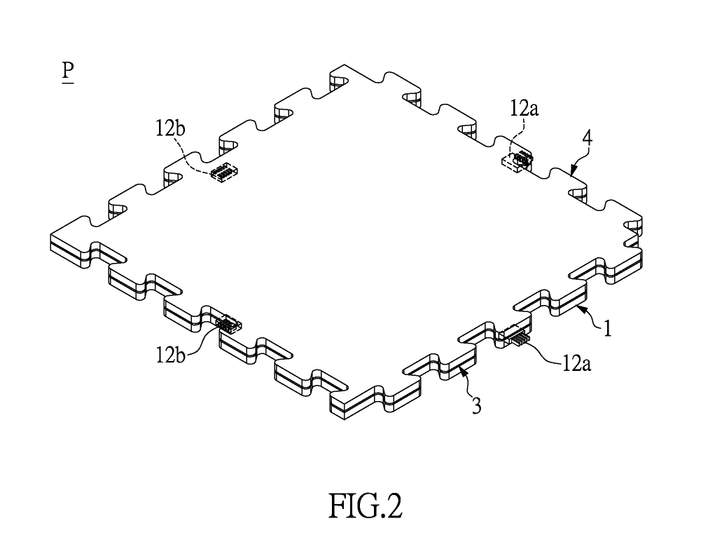

Please refer to FIGS. 1 and 2. FIG. 1 is an exploded view of the activity-sensing ground pad according to a preferred embodiment of the present invention. FIG. 2 is an assembled view of the activity-sensing ground pad according to a preferred embodiment of the present invention. The activity-sensing ground pad P, as shown in the Figures, includes sequentially a modular base 1, a sensing layer 2, an insulating layer 3, and a conductive elastic layer 4. Please note that the preferred embodiment is only for an illustrative purpose and is not meant to be a limitation of the present invention. For example, the activity-sensing ground pad P may include one of a triangle, a square, a rectangle, a rhombus, a parallelogram, and/or other shapes or configurations, to meet the requirements of indoor spatial applications.

Further, the activity-sensing ground pad P can be sized similarly to the commercial rubber ground pad, where the pad size is not restricted. The activity-sensing ground pad P can act as a thin-film switch, wherein the insulating layer 3 is configured to isolate the sensing layer 2 and the conductive elastic layer 4, and the conductive elastic layer 4 is configured to press against the sensing layer 2 by applying a force to the conductive elastic layer 4. The contact quality between the sensing layer 2 and the conductive elastic layer 4 is positively correlated to external force.

Specifically, the modular base 1, which depends on the shape, is formed with a pair of assembling sides. Said assembling sides, as shown in FIG. 1, are a first assembling side 10a and a second assembling side 10b opposite to the first assembling side 10a. The second assembling side 10b of the modular base 1 is provided for connecting the first assembling side 10a of another modular base 1. Thereby, at least two activity-sensing ground pads P can be assembled together.

Moreover, on the first assembling side 10a of the modular base 1, there are arranged a plurality of first engagement portions 11a, each of which can be, but not limited to, a block. On the second assembling side 10b of the modular base 1, there are arranged a plurality of second engagement portions 11b for coupling the first engagement portions 11a, each of which can be, but not limited to, a slot. It is worth to note that a first electrical connector 12a is arranged on one of the first engaging portions 11a, and a second electrical connector 12b is arranged on one of the second engaging portions 11b in correspondence with the first electrical connector 12a.

For the instant embodiment, the first electrical connector 12a is a male docking connector, and the second electrical connector 12b is a female docking connector. However, the instant embodiment is not limited thereto. For example, the first electrical connector 12a can be a male magnetic connector, and the second electrical connector 12b can be a female magnetic connector.

Please refer to FIGS. 1, 2 and 3. The sensing layer 2 is arranged on the modular base 1, having a plurality of sensing electrode modules 21 electrically connected to the first and second electrical connectors 12a, 12b. Specifically, the modular base 1 has an accommodating cavity, and the sensing layer 2 can be installed in the accommodating cavity by any suitable binding means, wherein the surface of the sensing layer 2 is flush with the surface of the modular base 1.

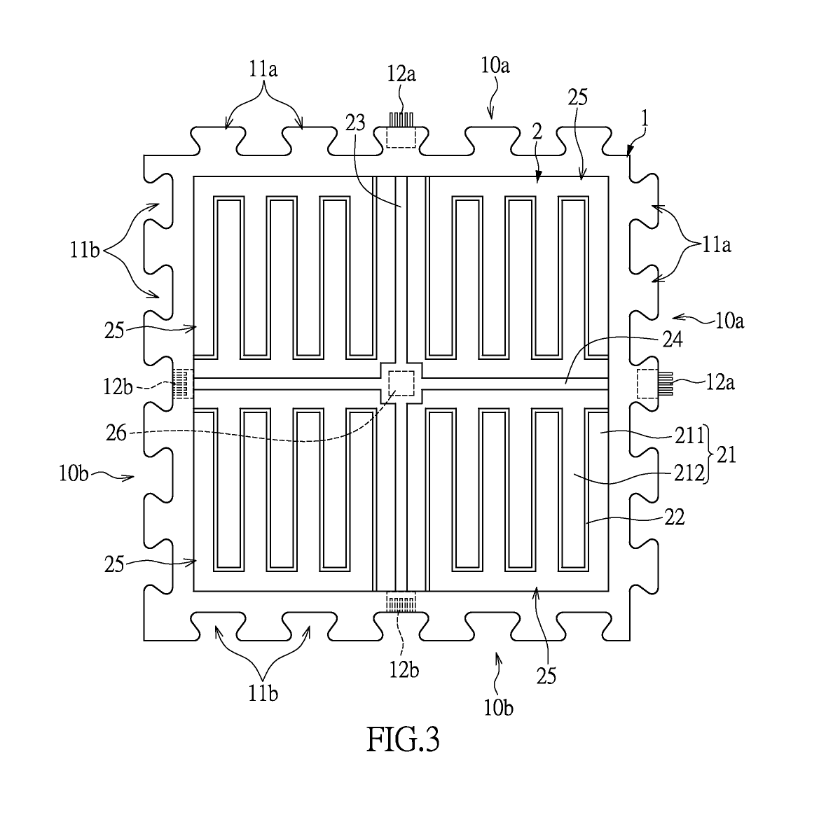

Specifically, each of the sensing electrode modules 21 consists of a first interdigitated electrode 211 served as a cathode and a second interdigitated electrode 212 served as an anode. The shape of the first interdigitated electrode 211 is corresponding to that of the second interdigitated electrode 212. Between the first and second interdigitated electrodes 211, 212 there is arranged a meander-like space 22 consisting of, as shown in FIG. 3, a series of U-shaped sections. Normally, the first and second interdigitated electrodes 211, 212 are insulated from each other.

To further explain in detail, the sensing layer 2 has at least one first-axial wire layout area 23 and at least one second-axial wire layout area 24 perpendicular to the at least one first-axial wire layout area 23. Between the first-axial and second-axial wire layout areas 23, 24 there are defined a plurality of sensing areas 25. The sensing electrode modules 21 are arranged in the sensing areas 25 respectively.

The activity-sensing ground pad P, for application to a monitoring system to monitor a home environment, has a processing unit 26. The processing unit 26, such as, but not limited to, a microprocessor, is configured to sense the occurrence of a touch event based on pressure. Specifically, the processing unit 26 of the activity-sensing ground pad P can generate a sensing signal in response to the touch event, and then transmit the sensing signal to another interlocked activity-sensing ground pad P. For the instant embodiment, the processing unit 26 is arranged at the junction of the first-axial and second-axial wire layout areas 23, 24, configured to electrically connect the sensing electrode modules 21 and the first and second electrical connectors 12a, 12b.

The layout of the first-axial and second-axial wire layout areas 23, 24 are adjustable according to the product requirements. However, any ground pad which includes a plurality of sensing electrode modules arranged therein and configured to electrically connect at least one docking-type connector via at least one wiring area is within the scope of the present invention.

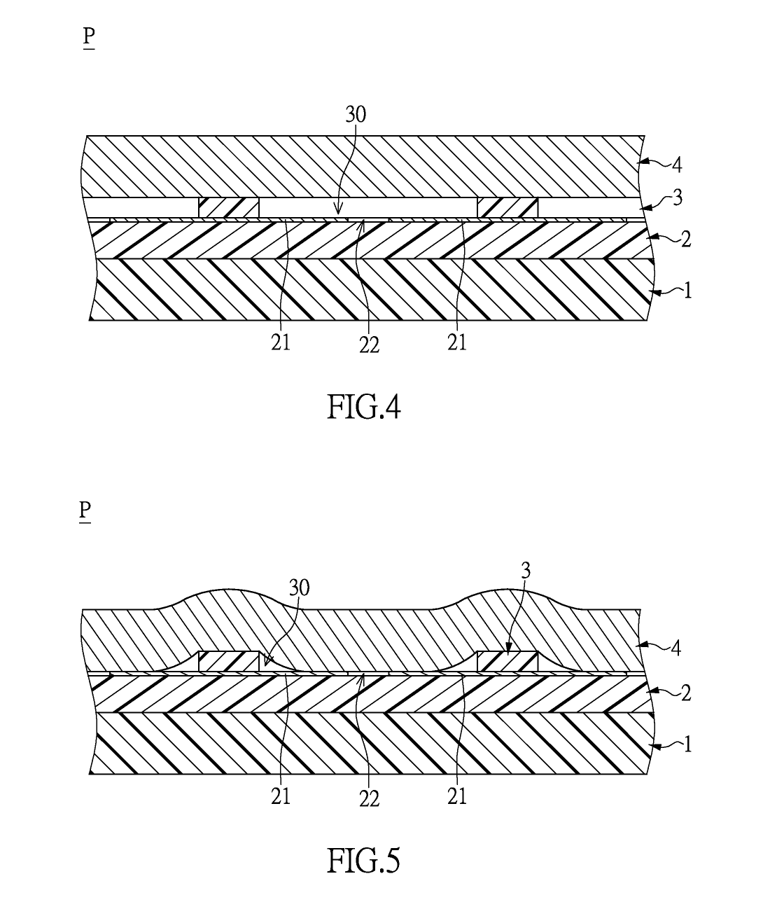

Please refer to FIGS. 1 and 2 as well as FIGS. 4 and 5. The insulating layer 3 is arranged on the sensing layer 2, and the conductive elastic layer 4 is arranged on the insulating layer 3. Specifically, the insulating layer 3 can be a foam layer having a predetermined thickness (preferably in a range between 1 cm and 4 cm. The conductive elastic layer 4 can be an equipotential layer of electrically conductive fabric, air cushion, or any other suitable elastic material, however it is not restricted thereto.

To further explain in detail, the insulating layer 3 is configured to temporarily separate the conductive elastic layer 4 and the sensing layer 2 without any external force existing. Thus, the error signal generated by contact between the conductive elastic layer 4 and the sensing layer 2 can be prevented. Moreover, in order to improve the contact quality of the conductive elastic layer 4 and the sensing layer 2 to enhance the accuracy and sensitivity of the sensing signal, the insulating layer 3 is formed with a plurality of through-holes 30 which are circular or rectangular in shape, however they are not restricted thereto. In practice a portion of the through-holes 30 are aligned with a meander-like space 22, wherein the diameter of each through-hole 30 must be larger than that of the meander-like space 22.

The following will describe the operating method of the activity-sensing ground pad P having the structural features as described in the above-mentioned aspect of the invention. As shown in FIG. 4, the conductive elastic layer 4 does not abut against the sensing layer 2 when no external force is being applied to operate the activity-sensing ground pad P in the circuit-breaking state. The resistance value detected by the processing unit 26 is nearly zero.

As shown in FIG. 5, the conductive elastic layer 4 is deformed under external force, wherein a portion of the conductive elastic layer 4 is pressed tightly against the sensing layer 2 through the through-holes 30 of the insulating layer 3 to operate the activity-sensing ground pad P in the open circuit state. The resistance value detected by the processing unit 26 is the serial or parallel resistance of the conductive elastic layer 4 and the sensing electrode modules 21 of the sensing layer 2.

Please note that, the total value of the serial or parallel resistance is positively correlated to the number of contacts and the tighter contact between the conductive elastic layer 4 and the corresponding sensing electrode modules 21 of the sensing layer 2. That is, the total value of the resistance variable according to the loading force as well as the loaded area can be determined, and in this way activity of the elderly person can be determined.

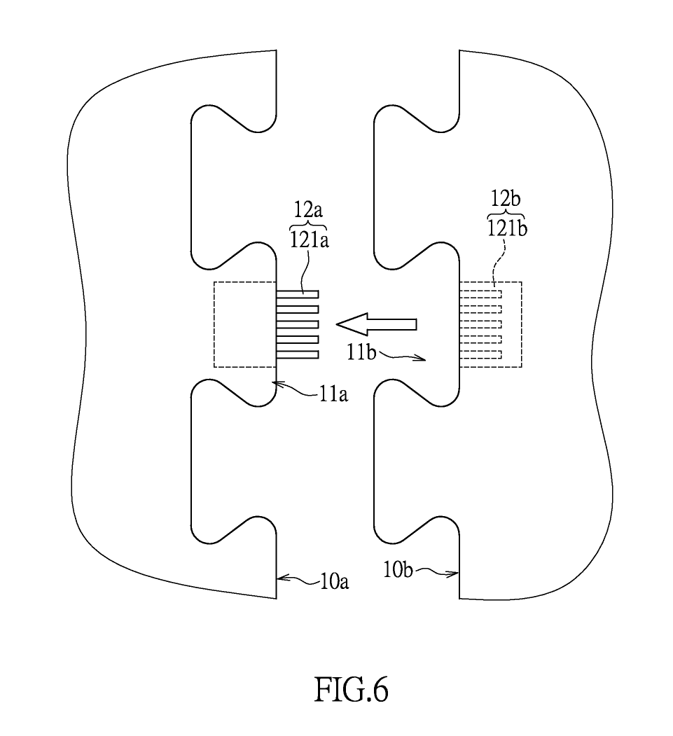

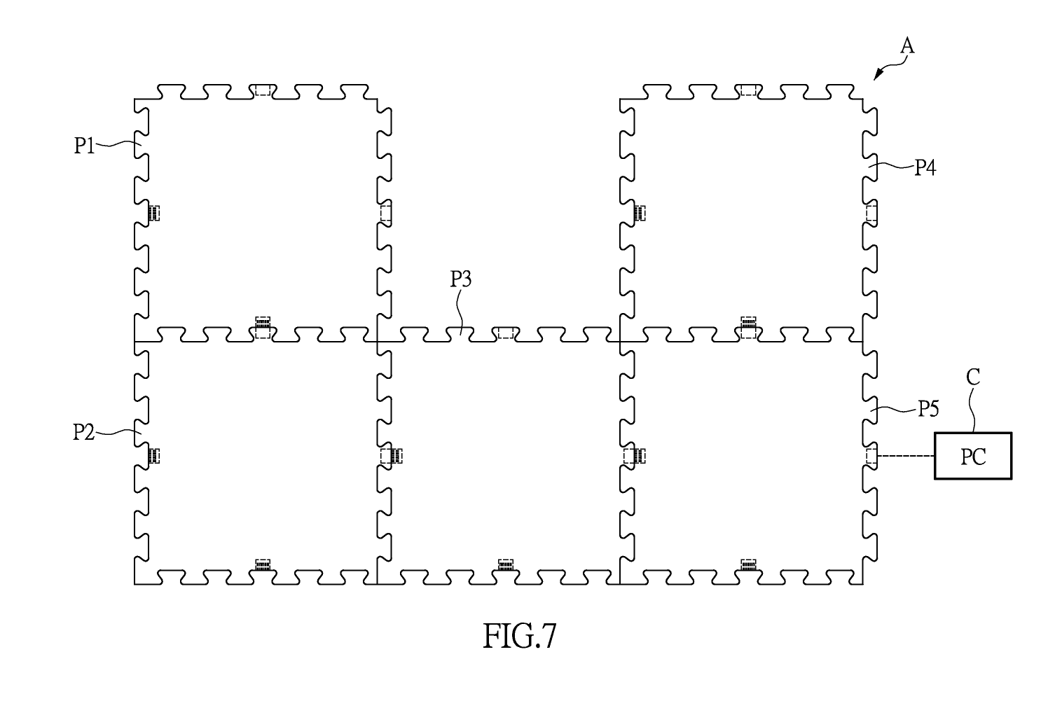

Please refer to FIGS. 6 and 7. The following will describe the application of the activity-sensing ground pad P having the structural features as described in the above-mentioned aspect of the invention. The monitoring system used in an in-house room environment, as shown in FIG. 7, includes an activity-sensing ground pad assembly A and a monitoring apparatus C.

Specifically, the activity-sensing ground pad assembly A1 consists of a plurality of activity-sensing ground pads P1-P5 of the same size which are aligned and interlocked together. For example, the second assembling side 10b of the activity-sensing ground pads P1 is connected to the first assembling side 10a of the activity-sensing ground pads P2 by joining the first and second engagement portions 11a, 11b together top down. In this manner, the second electrical connector 12b of the activity-sensing ground pads P1 is coupled with the first electrical connector 12a the activity-sensing ground pads P2, and so the activity-sensing ground pads P3-P5 can be linked together, which does not need to be more fully described.

For the instant embodiment, the first electrical connector 12a has a plurality of output terminals 121a which protrude out of the corresponding first engaging portion 11a. The second electrical connector 12b has a plurality of docking-type terminal slots 121b which are exposed to the corresponding second engaging portion 11b.

The monitoring apparatus C1 (i.e. a computer) is electrically connected to the activity-sensing ground pad assembly A1 in a wire/wireless way. A nurse or a personal care attendant can track the location of the activity-sensing ground pads P1-P5 and then analyze sensing signals via the monitoring apparatus C1, thereby judging the user's activity state (i.e. walking state, standing state, sitting state, falling down state, etc.). Further, said monitoring system can be integrated with a communication system to provide emergence medical treatment and health care. That is, the occurrence of accident events can be transmitted to nurses, personal care attendants, or his/her remote friends and relatives within a relatively short time.

For the instant embodiment, the monitoring apparatus C1 can be, but is not limited to, a single computer. In the various embodiments, the monitoring apparatus C1 can be an electronic device assembly which includes a plurality of computers, or include at least one computer and at least one smartphone/data solution device.

The present invention has the following advantages:

First, the activity-sensing ground pads apply human motion sensing technologies to ground pad structure, and are adapted to interlock with one another to meet the requirements of indoor spatial applications via the modular base and the docking-type connector. Thereby, the motion of the elderly, children, and persons with disabilities can be detected and tracked by an unconscious method and emergency health care treatment can be provided in a short period of time.

Second, the main body of the activity-sensing ground pad includes a sensing layer and an insulating layer, wherein the insulating layer is formed with a plurality of through-holes aligned with the meander-like space of the sensing layer. Therefore, efficient and accurate detection can be achieved.

Further, the monitoring system applying the activity-sensing ground pad assembly does not affect the user's daily life.

The descriptions illustrated supra set forth simply the preferred embodiments of the instant disclosure; however, the characteristics of the instant disclosure are by no means restricted thereto. All changes, alterations, or modifications conveniently considered by those skilled in the art are deemed to be encompassed within the scope of the instant disclosure delineated by the following claims.

What is claimed is:

1. An activity-sensing ground pad for interlocking with another activity-sensing ground pad, comprising:
a modular base having a first assembling side with a plurality of first engagement portions thereon and a second assembling side opposite to the first assembling side with a plurality of second engagement portions thereon, wherein a first electrical connector is arranged on one of the first engaging portions, and a second electrical connector is arranged on one of the second engaging portions in correspondence with the first electrical connector;
a sensing layer arranged on the modular base, having a plurality of sensing electrode modules electrically connected to the first and second electrical connectors, and each of which is formed with a channel;
an insulating layer arranged on the sensing layer, having a plurality of through-holes, and a portion of which are aligned with the channel; and
a conductive elastic layer formed to cover the insulating layer.

2. The activity-sensing ground pad according to claim 1, wherein the sensing layer has at least one first-axial wire layout area, at least one second-axial wire layout area perpendicular to the at least one first-axial wire layout area, and a plurality of sensing areas between the at least one first-axial and second-axial wire layout areas, and the sensing electrode modules are arranged in the sensing areas respectively.

3. The activity-sensing ground pad according to claim 2, wherein each of the sensing electrode modules includes a first interdigitated electrode and a second interdigitated electrode corresponding the first interdigitated electrode, and between the first and second interdigitated electrodes there is a meander channel.

4. The activity-sensing ground pad according to claim 2, wherein the sensing layer has a processing unit arranged thereon at the junction of the at least one first-axial and second-axial wire layout areas, configured to electrically connect the first and second electrical connectors via the at least one first-axial and second-axial wire layout areas.

5. The activity-sensing ground pad according to claim 1, wherein the first electrical connector has a plurality of output terminals that protrude out from the corresponding first engagement portion, the second electrical connector has a plurality of docking-type terminal slots coupled to the output terminals.

6. An activity-sensing ground pad assembly for integrating with a monitoring apparatus to monitor human activity, comprising at least two activity-sensing ground pads, each of which comprises:
a modular base having a first assembling side with a plurality of first engagement portions thereon and a second assembling side opposite to the first assembling side with a plurality of second engagement portions thereon, wherein a first electrical connector is arranged on one of the engaging portions, and a second electrical connector is arranged on one of the engaging portions in correspondence with the first electrical connector;
a sensing layer arranged on the modular base, having a plurality of sensing electrode modules electrically connected to the first and second electrical connectors, and each of which is formed with a channel;
an insulating layer arranged on the sensing layer, having a plurality of through-holes, and a portion of which are aligned with the channel; and
a conductive elastic layer formed to cover the insulating layer.

7. The activity-sensing ground pad assembly according to claim 6, wherein the sensing layer has at least one first-axial wire layout area, at least one second-axial wire layout area perpendicular to the at least one first-axial wire layout area, and a plurality of sensing areas between the at least one first-axial and second-axial wire layout areas, and the sensing electrode modules are arranged in the sensing areas respectively.

8. The activity-sensing ground pad assembly according to claim 7, wherein each of the sensing electrode modules includes a first interdigitated electrode and a second interdigitated electrode corresponding the first interdigitated electrode, and between the first and second interdigitated electrodes there is a meander channel.

9. The activity-sensing ground pad assembly according to claim 7, wherein the sensing layer has a processing unit arranged thereon at the junction of the at least one first-axial and second-axial wire layout areas, configured to electrically connect the first and second electrical connectors via the at least one first-axial and second-axial wire layout areas.

10. The activity-sensing ground pad according to claim 6, wherein the first electrical connector has a plurality of output terminals that protrude out from the corresponding first engagement portion, the second electrical connector has a plurality of docking-type terminal slots coupled to the output terminals.
